(19) United States
(12) Patent Application Publication
(10) Pub. No.: US 2009/0291021 A1
(43) Pub. Date: Nov. 26, 2009

(54) Methods for Preventing Microbial Colonization of Gas Cylinders and Coupling Components

(75) Inventor: Jeffrey H. Ping, Braselton, GA (US)

Correspondence Address: SUTHERLAND ASBILL & BRENNAN LLP, 999 PEACHTREE STREET, N.E., ATLANTA, GA, 30309, US

(73) Assignee: THE BOC GROUP, INC., Murray Hill, NJ (US)

(21) Appl. No.: 12/512,743

(22) Filed: Jul. 30, 2009

Related U.S. Application Data
(62) Division of application No. 11/570,817, filed on Nov. 27, 2007.
(60) Provisional application No. 60/581,220, filed on Jun. 18, 2004.

(51) Int. Cl. A61L 2/18; A61J 1/00

(52) U.S. Cl. 422/28; 220/586

(57) ABSTRACT

This invention relates to the field of gas-containing storage vessels, and more specifically to the provision for antimicrobial surfaces within such vessels and in the connecting hardware associated with various applications of such vessels, so that microbial colonization of the interior of such vessels may be eliminated or retarded. This antimicrobial feature may result in improved safety in the use of such vessels, with reduced risk of the transmission of infection to a user. The invention further includes methods to provide gas-containing storage vessels with antimicrobial surfaces, so that microbial colonization of the interior of such vessels may be eliminated or retarded.

### TECHNICAL FIELD OF THE INVENTION

The present invention relates generally to the field of containers, couplings, related in-line devices, and delivery conduits for gases used in respiratory support applications, and relates more specifically to the lining employed within such containers, couplings, related in-line devices, and delivery conduits, and particularly to such linings in which an anti-microbial agent or quality is incorporated to retard the growth or transmission of microbes therewithin.

### BACKGROUND OF THE INVENTION

In industrial, healthcare, aerospace, and recreational underwater settings, a gas or mixture of gases is often contained within pressurized cylinders, tanks, or other containers, from which a controlled release of the gas is effected for a desired purpose. In many such applications, compressed air, pure oxygen, or mixtures of oxygen and other gases is often contained within pressurized cylinders, tanks, or other vessels and dispensed for use in breathing by persons in low oxygen environments, or by persons with impaired respiratory function.

In an application where a person is relying upon a pressurized gas container to provide oxygen for respiratory assistance or support, there is the potential risk that a pathogenic contaminant within the container might be inhaled by the person, with the potential transmission of disease. Specifically, it is possible that a pressurized gas container might contain pathogenic microorganisms that could be introduced during the process of filling the container with gas. These microorganisms might then, in whole or in part, be blown out, under pressure, as the container is used, and might then pass into the lungs of a user on inhalation, causing pneumonitis, lung abscess and/or other respiratory or mucosal infections or irritations.

Existing technology for pressurized gas cylinders, tanks, and other containers does not provide for the inclusion of antimicrobial linings therewithin to reduce the chance of infectious microbes with inspired air.

Thus, the need exists for pressurized gas cylinders, tanks, and other containers used for biological respiratory support that incorporate a lining with intrinsic antimicrobial properties.

The need further exists for pressure regulators and other devices which couple to such pressurized gas cylinders, tanks, and other containers when used in respiratory support applications to similarly be provided with antimicrobial linings to reduce the chance of the introduction of infectious microbes with inspired air or gas.

It is well known that colonization of bacteria on the surfaces of medical implants or other parts of some medical devices can produce serious health problems, including the need to remove and/or replace an implanted device and to vigorously treat secondary infective conditions. A considerable amount of attention and study has been directed toward preventing such colonization by the use of antimicrobial agents, such as antibiotics, bound to the surface of the materials employed in such devices.

Various methods have previously been employed to contact or coat the surfaces of certain medical devices with an antimicrobial agent. However, while gas cylinders, tanks, and other containers may be used in both medical and non-medical applications, no known prior uses of antimicrobial linings or coatings have been directed to the linings of such containers, or to the interior surfaces of the valves and regulators which connect thereto.

These and many other methods of coating various medical devices with antibiotics or antimicrobial properties appear in numerous patents and medical journal articles. Practice of many of the prior art coating methods results in a catheter or other medical item wherein only the surface of the device is coated with an antibiotic. While the surface coated item does provide effective protection against bacteria initially, the effectiveness of the coating diminishes over time. During use of the medical item, the antimicrobials may leach from the surface of the device into the surrounding environment. Over a period of time, the amount of antibiotics present on the surface may decrease to a point where the protection against bacteria is no longer effective.

While some types of medical devices and other items may be readily amenable to replenishing antibiotics within a lining or coating, gas containers are generally not accessible for internal applications of liquids, and neither routine drying nor removal of liquid or biologic residue within the pressurized gas container is practical. Therefore, it would be desirable in a gas container to provide a lining with antimicrobial properties that either are longlasting or capable of replenishment within a pressurized, gaseous environment.

### SUMMARY OF THE INVENTION

It is an object according to the present invention to provide gas containers with an antimicrobial lining or other antimicrobial properties to prevent the potential colonization of pathogenic microbes within said containers.

It is a further object according to the present invention to provide gas valves, regulators, and related connectors with an antimicrobial lining or other antimicrobial properties to prevent the potential colonization of pathogenic microbes within said valves, regulators, and related connectors.

In various embodiments according to the present invention, the antimicrobial properties provided within gas containers, valves, regulators, and related connectors may be derived from applications of known antibiotic pharmacologic agents.

In yet other various embodiments according to the present invention, the antimicrobial properties provided within gas containers, valves, regulators, and related connectors may be derived from materials intrinsically bonded within the lining or wall structural materials for said gas containers, valves, regulators, and related connectors.

In still other various embodiments according to the present invention, the antimicrobial properties provided within gas containers, valves, regulators, and related connectors may be derived from materials coating or bonded to the surface of lining or wall structural materials for said gas containers, valves, regulators, and related connectors.

It is yet a further object according to the present invention to provide gas valves, regulators, and related connectors with an antimicrobial lining or other antimicrobial properties to prevent the potential colonization by pathogenic or other gram positive bacteria within said valves, regulators, and related connectors.

It is yet a further object according to the present invention to provide gas valves, regulators, and related connectors with an antimicrobial lining or other antimicrobial properties to prevent the potential colonization by pathogenic or other gram negative bacteria within said valves, regulators, and related connectors.

It is yet a further object according to the present invention to provide gas valves, regulators, and related connectors with an antimicrobial lining or other antimicrobial properties to prevent the potential colonization by pathogenic or other fungi within said valves, regulators, and related connectors.

It is yet a further object according to the present invention to provide gas valves, regulators, and related connectors with an antimicrobial lining or other antimicrobial properties to prevent the potential colonization by pathogenic or other viruses within said valves, regulators, and related connectors.

These and other features, aspects, and other advantages according to the present invention will become more apparent and more readily understood with regard to the following specification, drawings, description, appended claims, and any examples of the present preferred embodiments of the invention which are disclosed herein.

### BRIEF DESCRIPTION OF THE DRAWINGS

FIG. 1 provides a sectional drawing of an exemplary gas cylinder containing an antimicrobial lining according to the present invention.

FIG. 2 provides a drawing of an exemplary gas regulator and connectors containing an antimicrobial lining according to the present invention.

### DETAILED DESCRIPTION OF THE INVENTION

The present invention may be understood more readily by reference to the following detailed description of the preferred embodiments of the invention and the Examples included herein. However, before the preferred embodiments of the devices and methods according to the present invention are disclosed and described, it is to be understood that this invention is not limited to the exemplary embodiments described within this disclosure, and the numerous modifications and variations therein that will be apparent to those skilled in the art remain within the scope of the invention disclosed herein. It is also to be understood that the terminology used herein is for the purpose of describing specific embodiments only and is not intended to be limiting.

Unless otherwise noted, the terms used herein are to be understood according to conventional usage by those of ordinary skill in the relevant art. In addition to the definitions of terms provided below, it is to be understood that as used in the specification and in the claims, “a” or “an” can mean one or more, depending upon the context in which it is used.

The term “gas container” as used herein is defined as any cylinder, tank, or other vessel used to confine and contain a gas for controlled release and use thereof. Preferably as gas container is capable of storing gas under high pressure.

The term “component” as used herein is defined as any gas valve, regulator, or other flow-through connector or attachment used to control the release and/or delivery of a gas from a container.

The term “coating” as used herein is defined as a layer of material that may be used to cover the interior surface of any container or component. A coating according to the present invention may be applied to the surface of the container or component by painting, spraying, electrodeposition, or any other known coating process, or such a coating may be impregnated within the material that forms the interior wall of the container or component. A coating according to the present invention shall be chemically inert or otherwise non-reactive with regard to the specific gas contained within the container having the coating. Moreover, a coating according to the present invention shall be non-toxic to human or other mammalian users.

The term “antimicrobial agent” as used herein is defined as any antiseptic, an antibiotic, or other substance or material or combination thereof that inhibits the growth or sustenance of microorganisms.

The term “antiseptic” as used herein is defined as a material that inhibits the growth or sustenance of microorganisms, including but not limited to alpha-terpineol, methylisothiazolone, cetylpyridinium chloride, chloroxyleneol, hexachlorophene, chlorhexidine and other cationic biguanides, methylene chloride, iodine and iodophores, triclosan, taurinamides, nitrofarantoin, methenamine, aldehydes, azylic acid, silver, other silver salts, silver benzyl peroxide, alcohols, metals and metal salts and acids, and carboxylic acids and salts.

One skilled in the art is cognizant that these antiseptics can be used in combinations of two or more to obtain a synergistic effect. Furthermore, the antiseptics may be dispersed along the surface of a container.

Some examples of combinations of antimicrobial agents include a mixture of chlorhexidine, chlorhexidine and chloroxylenol, chlorhexidine and methylisothiazolone, chlorhexidine and alpha-terpineol, methylisothiazolone and alpha-terpineol; thymol and chloroxylenol; chlorhexidine and cetylpyridinium chloride; or chlorhexidine, methylisothiazolone and thymol. These combinations provide a broad spectrum of activity against a wide variety of organisms.

The term “antibiotics” as used herein is defined as a substance that inhibits the growth of microorganisms. For example, the antibiotic may inhibit cell wall synthesis, protein synthesis, nucleic acid synthesis, or alter cell membrane function.

Classes of antibiotics that can be used include, but are not limited to, macrolides (i.e., erythromycin), penicillins (i.e., nafcillin), cephalosporins (i.e., cefazolin), carbepenems (i.e., imipenem, aztreonam), other beta-lactam antibiotics, beta-lactam inhibitors (i.e., sulbactam), oxalines (i.e. linezolid), aminoglycosides (i.e., gentamicin), chloramphenicol, sulfonamides (i.e., sulfamethoxazole), glycopeptides (i.e., vancomycin), quinolones (i.e., ciprofloxacin), tetracyclines (i.e., minocycline), fusidic acid, trimethoprim, metronidazole, clindamycin, mupirocin, rifamycins (i.e., rifampin), streptogramins (i.e., quinupristin and dalfopristin) lipoprotein (i.e., daptomycin), polyenes (i.e., amphotericin B), azoles (i.e., fluconazole), and echinocandins (i.e., caspofungin acetate).

Examples of specific antibiotics that can be used include, but are not limited to, erythromycin, nafcillin, cefazolin, imipenem, aztreonam, gentamicin, sulfamethoxazole, vancomycin, ciprofloxacin, trimethoprim, rifampin, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linezolid, temafloxacin, tosufloxacin, clinafloxacin, sulbactam, clavulanic acid, amphotericin B, fluconazole, itraconazole, ketoconazole, and nystatin. Other examples of antibiotics, such as those listed in Sakamoto et al, U.S. Pat. No. 4,642,104 herein incorporated by reference will readily suggest themselves to those of ordinary skill in the art.

The term “bacterial interference” as used herein is defined as an antagonistic interactions among bacteria to establish themselves and dominate their environment. Bacterial interference operates through several mechanisms, i.e., production of antagonistic substances, changes in the bacterial microenvironment, and reduction of needed nutritional substances.

The term “effective concentration” means that a sufficient amount of the antimicrobial agent is added to decrease, prevent or inhibit the growth of bacterial and/or fungal organisms. The amount will vary for each compound and upon known factors such as pharmaceutical characteristics; the type of medical device; age, sex, health and weight of the recipient; and the use and length of use. It is within the skilled artisan's ability to relatively easily determine an effective concentration for each compound.

The term “gram-negative bacteria” or “gram-negative bacterium” as used herein is defined as bacteria which have been classified by the Gram stain as having a red stain. Gram-negative bacteria have thin walled cell membranes consisting of a single layer of peptidoglycan and an outer layer of lipopolysacchacide, lipoprotein, and phospholipid. Exemplary organisms include, but are not limited to, Enterobacteriacea consisting of Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary gram-negative organisms not in the family Enterobacteriacea include, but are not limited to, Pseudomonas aeruginosa, Stenotrophomonas maltophilia, Burkholderia, Cepacia, Gardenerella, Vaginalis, and Acinetobacter species.

The term “gram-positive bacteria” or “gram-positive bacterium” as used herein refers to bacteria, which have been classified using the Gram stain as having a blue stain. Gram-positive bacteria have a thick cell membrane consisting of multiple layers of peptidoglycan and an outside layer of teichoic acid. Exemplary organisms include, but are not limited to, Staphylococcus aureus, coagulase-negative staphylococci, streptococci, enterococci, corynebacteria, and Bacillus species.

The term “mutant” as defined herein refers to a bacterium that has been mutated using standard mutagenesis techniques such as site-directed mutagenesis. One skilled in the art recognizes that the term mutant includes, but is not limited to base changes, truncations, deletions or insertions of the wild-type bacterium. Thus, the size of the mutant bacterium may be larger or smaller than the wild-type or native bacterium. Yet further, one skilled in the art realizes that the term mutant also includes different strains of bacteria or bacteria that has been chemically or physically modified as used herein.

The term “non-pathogenic bacteria” or “non-pathogenic bacterium” includes all known and unknown non-pathogenic bacterium (gram positive or gram negative) and any pathogenic bacteria that has been mutated or converted to a non-pathogenic bacterium. Furthermore, a skilled artisan recognizes that some bacteria may be pathogenic to specific species and non-pathogenic to other species; thus, these bacteria can be utilized in the species in which it is non-pathogenic or mutated so that it is non-pathogenic.

One specific embodiment of the present invention is a method for coating the interior of a container comprising the steps of applying to at least a portion of the surface of said container, an antimicrobial coating layer, wherein said antimicrobial coating layer comprises an antimicrobial agent in an effective concentration to inhibit the growth of bacterial and fungal organisms relative to uncoated containers; and applying to at least a portion of the surface of said container, a non-pathogenic bacterial coating layer, wherein said non-pathogenic bacterial coating layer comprises a non-pathogenic gram-negative bacterium in an effective concentration to inhibit the growth of pathogenic bacterial and fungal organisms, wherein said non-pathogenic gram-negative bacterium is resistant to said antimicrobial agent.

The linings or interior walls of containers that are amenable to impregnation by the antimicrobial combinations are generally comprised of a non-metallic material such as thermoplastic or polymeric materials. Examples of such materials are rubber, plastic, polyethylene, polyurethane, silicone, Gortex (polytetrafluoroethylene), Dacron (polyethylene tetraphthalate), polyvinyl chloride, Teflon (polytetrafluoroethylene), latex, elastomers, nylon and Dacron sealed with gelatin, collagen or albumin.

The amount of each antimicrobial agent used to coat an interior container wall may vary to some extent, but is at least a sufficient amount to form an effective concentration to inhibit the growth of bacterial and fungal organisms. The antimicrobial agent may be applied to the interior surface wall of a container in a variety of methods. Exemplary application methods include, but are not limited to, spraying, painting, dipping, sponging, atomizing, bonding, smearing, impregnating and spreading.

A skilled artisan is cognizant that the development of microorganisms in culture media is dependent upon a number of very important factors, e.g., the proper nutrients must be available; oxygen or other gases must be available as required; a certain degree of moisture is necessary; the media must be of the proper reaction; proper temperature relations must prevail; the media must be sterile; and contamination must be prevented.

A satisfactory microbiological culture contains available sources of hydrogen donors and acceptors, carbon, nitrogen, sulfur, phosphorus, inorganic salts, and, in certain cases, vitamins or other growth promoting substances. The addition of peptone provides a readily available source of nitrogen and carbon. Furthermore, different media results in different growth rates and different stationary phase densities. A rich media results in a short doubling time and higher cell density at a stationary phase. Minimal media results in slow growth and low final cell densities. Efficient agitation and aeration increases final cell densities. A skilled artisan will be able to determine which type of media is best suited to culture a specific type of microorganism. For example, since 1927, the DIFCO manual has been used in the art as a guide for culture media and nutritive agents for microbiology.

Similarly, if one is to retard or prevent the growth of unwanted colonies of microorganisms within gas containers, the same fators necessary for microbial growth must be eliminated or controlled.

In one specific embodiment according to the present invention, a gas container is provided with an interior antimicrobial coating layer to inhibit the growth of bacterial and fungal organisms relative to an uncoated gas container.

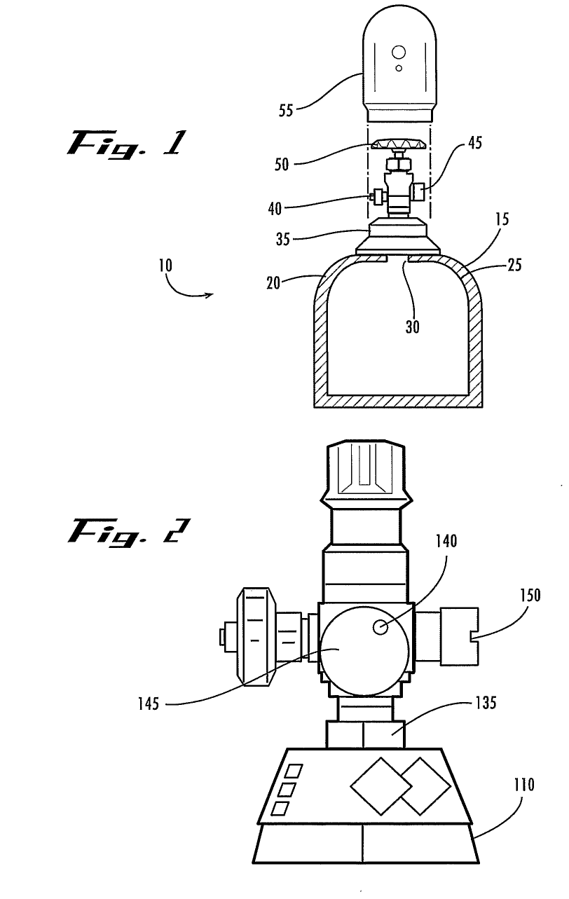

Referring now to an embodiment according to the present invention as shown in FIG. 1, a gas container 10 is provided in the form of a cylindrical tank, comprising tank walls 15 with an outer tank surface 20 and an interior tank surface 25, and at least one tank portal 30. The tank portal 30 is further provided with a tank connector 35 and a tank valve 40, so that a gas may be introduced into the container 10 under pressure through said tank valve 40, tank connector 35, and tank portal 30, and then retained within said container 10 by closing said tank valve 40. The tank valve 40 is opened or closed by operation of a valve control 50 by a user. The tank valve 40 is further provided with a least one external port 45 through which gas within the gas container 10 may either be dispensed or refilled. The tank connector 35 serves to attach the tank valve 40 to the tank portal 30, and may be removable to allow physical access to the interior tank surface 25 for cleaning or maintenance within. The gas container 10 may further be provided with a tank cap 55 to cover and protect the tank valve 40 when the gas container 10 is not in use.

In the embodiment according to the present invention shown in FIG. 1, the interior tank surface 25 may be provided with an antimicrobial coating (not shown) that adheres directly to the interior tank surface 25. In alternate embodiments according to the present invention, the interior tank surface 25 may be provided with an intermediate coating (not shown) that adheres directly to the interior tank surface 25 and then serves to receive an antimicrobial coating (not shown) that may adhere or be bonded directly to the intermediate coating. Such an intermediate coating may be a metallic coating or a polymer, capable of being firmly adherent to the interior tank surface 25, and further capable of receiving and retaining an antimicrobial coating (not shown).

In various embodiments according to the present invention, the inner tank surface 25 may be constructed of metal, metal alloy, ceramic, plastic, other polymers, or any combination(s) of the preceding materials.

Coatings may be applied to the inner tank surface 25 using any conventional coating process, including, but not limited to, painting, immersion, spraying, ionic deposition, electron deposition, sputter deposition, or any other coating method.

In such embodiments according to the present invention as described above, the interior tank surface 25 may be treated or re-treated at intervals to replenish the antimicrobial coating. This may be accomplished during the process of refilling or recharging the gas content, and may further involve cleaning the old coating with a suitable solvent, then rinsing and drying the tank interior, and then re-applying the antimicrobial coating, removing any excess, and drying the tank interior before gas is refilled into the tank for use.

In still other embodiments according to the present invention, the interior tank surface 25 may be provided with a metallic coating that may have inherent antimicrobial properties, such as various organic and inorganic substances, including silver, titanium, copper, cobalt, magnesium, and other metal salts. Alternately, other embodiments according to the present invention may employ materials which comprise the tank wall that inherently have such antimicrobial properties, such that the antimicrobial properties become an integral part of the structural wall of the tank. In such settings, the antimicrobial capabilities of the tank may be longlasting, and may or may not require periodic rejuvenation from instilled agents during cleaning/refill operations.

The gas containers according to the invention can be fabricated from a wide variety of substrate materials, with the primary materials considerations being sufficient strength to withstand necessary internal pressures, chemical non-reactivity with respect to the contained gas, and weight considerations dictated by the specific application. Such materials include metals metal alloys, ceramics, plastics, other polymers, and any combinations thereof.

Such metallic materials for gas containers according to the present invention include, but are not limited to, iron, steel, stainless steel, nickel, titanium, manganese, and aluminum.

Potential structural ceramics include compositions of inorganic elements, such as nitrides, borides, carbides, suicides, oxides, and mixtures thereof. Ceramics also include glasses, glass ceramics, oxide ceramics, and other partially crystalline inorganic materials.

Potential structural plastics for gas containers include addition polymers, polycondensation products, and polyaddition compounds. Specific examples include polyolefins, such as polyethylene and polypropylene; copolymers of ethylene and propylene with one another and/or with other olefinically unsaturated monomers, such as 1-butene, vinyl acetate and acrylonitrile; polyesters, such as polyethylene terephthalate and polybutylene terephthalate; polycarbonates; polyamides, such as polycaprolactam and polylaurolactam; polyalkylene fluorides, such as polyvinylidene fluoride and polytetrafluoroethylene; and polyurethanes.

Articles of the present invention may also be made of a combination of the above mentioned metals, ceramics, polymers, and plastics.

Antimicrobial agents are chemical compositions that inhibit microbial growth or kill bacteria, fungi and other microorganisms. Different inorganic and organic substances display antimicrobial activity. Among the simple organic substances that possess antimicrobial activity are carboxylic acids, alcohols and aldehydes, most of which appear to act by protein precipitation or by disruption of microbial cell membrane.

The antimicrobial activity of inorganic substances is generally related to the ions, toxic to other microorganisms, into which they dissociate. The antimicrobial activity of various metal ions, for example, is often attributed to their affinity for protein material and the insolubility of the metal proteinate formed. Metal-containing salts are thus among the inorganic substances that act as antimicrobial agents.

Metal inorganic salts, including simple salts of metal cations and inorganic anions like silver nitrate, are often soluble and dissociable and, hence, offer ready availability of potentially toxic ions. But such salts may be quickly rendered ineffective as antimicrobial agents by the combining of the metal ion with extraneous organic matter or with anions from tissue or bodily fluid. As a consequence, prolonged or controlled bacteriostatic and bacteriocidal activity is lost.

Metal salts or complexes of organic moieties such as organic acids, on the other hand, are often less soluble and, therefore, are less dissociable than the soluble metal inorganic salts. Metal organic salts or complexes generally have a greater stability with respect to extraneous organic matter, and anions present in the environment of the living cell than metal inorganic salts, but have less toxic potential by virtue of their greater stability. The use of heavy metal ions with polyfunctional organic ligands as antimicrobial agents has been disclosed, for example, in U.S. Pat. No. 4,055,655.

The silver ion is an example of a metal ion known to possess antimicrobial activity. The use of silver salts, including both inorganic and organic ligands, as antimicrobial agents has long been known in the prior art. The dissociation of the silver salt provides silver ions which provide the antimicrobial activity. Silver ions react with a variety of anions as well as with chemical moieties of proteins. Precipitation of proteins, causing disruption of the microbial cell membrane and complexation with DNA, is likely the basis of the antimicrobial activity. Silver ions in high concentration will form insoluble silver chloride and thereby deplete chloride ions in vivo.

In an exemplary embodiment according to the invention, pressurized gas containers are imparted with antimicrobial containment properties by coating the substrate of the interior tank surfaces with cyanoalkylated hydroxyalkylcellulose. A gas container is first opened at the tank portal to provide access to the tank's interior. Coatings may then be applied by any conventional coating technique such as dipping, spraying or spreading. Typically, cyanoalkylated hydroxyalkylcellulose is dissolved in a volatile solvent, such as acetone, and coated onto the substrate. The solvent evaporates at, or slightly above, room temperature, leaving cyanoalkylated hydroxyalkylcellulose coating on the substrate surface.

The resistance of the article to microbial growth is highest when the coating is completely smooth and pore-free. An smooth, pore-free coating is most easily produced when the underlying substrate is also smooth and pore-free. Interior tank surfaces with smooth, pore-free substrates are therefore preferred, and may be prepared by polishing and or plating the tank interior surface using conventional metal polishing and plating techniques.

A cyanoalkylated hydroxyalkylcellulose coating is hydrophobic and insoluble in water, but it can absorb water and swell, depending on the degree of cyanoalkylation. The coating can be modified so it will no longer absorb water, and will no longer be soluble in organic solvents like acetone. This modification involves exposing the coated article to a plasma treatment or corona discharge, or to high-energy radiation. High-energy radiation is defined here to mean radiation more energetic than visible light, and includes UV rays, X-rays, and radiation generated by electron beams. The preferred method to modify the cyanoalkylated hydroxyalkylcellulose coating is to expose it to UV radiation.

The modified coatings have better adhesion to the underlying substrate than unmodified coatings, especially on smooth, pore-free substrates. The antimicrobial properties, the desired low coefficient of friction, and the low toxicity of the coatings are not diminished by their modification.

The antimicrobial coating composition in another embodiment according to the present invention may comprise a metal-containing sulfonylurea compound, along with one or both of a water-soluble and a water-insoluble carboxylic acid compound, in a polymeric matrix. A single coating of the composition can provide antimicrobial activity.

Sulfonylurea compounds that are suitable for use in accordance with the present invention include acetohexamide, tolazamide and chloropropamide. A representative metal-containing sulfonylurea compound suitable for use in the present invention is silver tolbutamide (AgTol), a white compound formed when equal molar amounts of silver nitrate and sodium tolbutamide, both in aqueous solution, are mixed. AgTol incorporates a tolbutamide ligand that is a sulfonylurea, tolbutamide.

The sulfonylureas are known for their hypoglycemic properties, but none are reported to be antimicrobial. Accordingly, tolbutamide is understood not to contribute any antimicrobial activity to silver tolbutamide, in contrast to the sulfadiazine component of silver sulfadiazine.

AgTol has a medium value dissociation constant estimated to be greater then pK=3.3. It does not deplete chloride from tissue fluid, but is soluble in a variety of organic solvents, including solvents containing polymers. The solubility of AgTol, which is not a polymer, is considerably greater than that of silver sulfadiazine. AgTol is not photostable when present in a coating, yet is observed to be light stable as a solid. The light instability of AgTol appears to be related both to the lack of stabilization of the silver ion in the compound and the nonpolymeric nature of AgTol.

Silver salts are typically light sensitive, and this photoinstability affects their use in many applications. However, in an application according to the present invention, the silver salts are generally used within the confines of an opaque, pressurized gas tank or other container, where photosensitivity is generally not relevant for consideration.

Thus, one antimicrobial coating in an embodiment according to the present invention may include a metal-containing sulfonylurea, preferably AgTol, and at least one of a water-soluble carboxylic acid and a water-insoluble carboxylic acid in a polymer matrix. The polymer material forming the matrix should permit suitable diffusion of the metal ions out of the matrix. An acceptable permeability is reflected, for example, in a high moisture-vapor transmission (MVTR) value, preferably in the range of about 100 to 2500 g/m.sup.2/24 hours/mil of membrane thickness. Polymers that can be used in this context include polyurethane, polyvinylchloride, nylon, polystyrene, polyethylene, polyvinyl alcohol, polyvinyl acetatae, silicone and polyester.

Exemplary of solvents which can be employed in the present invention are those characterized by a solubility parameter, expressed in terms of (Cal/cn.sup.2).sup.½, of between about 9 and 12, such as (Cal/cm2) tetrahydrofuran, benzene, diacetone alcohol, methyl ethyl ketone, acetone and N-methylpyrrolidone.

A variety of water-insoluble carboxylic acids are conveniently employed in the present invention, including fatty acids, such as stearic acid, capric acid, lauric acid, myrisic acid, palmitic acid and arachidic acid, as well as cholic acid, deoxycholic acid, taurocholic acid and glycocholic acid. By the same token, numerous water-soluble carboxylic acids are suitable, such as citric acid, gluconic acid, glutamic acid, glucoheptonic acid, acetic acid, propionic acid and butyric acid.

The molar amount of each type of carboxylic acid can be varied, preferably from about 0 to about 2 mole per mole of metal-containing sulfonylurea. The respective amounts used of water-soluble and water-insoluble acids will depend upon the level of antimicrobial activity desired from the coating.

The coating can be applied to a medical device by dipping in the antimicrobial solution and thereafter allowing the solvent to evaporated. Both inside and outside surfaces can be coated. Alternatively, the medical articles can be sprayed with the mixture and the solvent allowed to evaporated. Likewise, the medical device can be painted with the solution, and the solvent allowed to evaporate. All coating processes can be carried out at room temperature, but evaporation of solvent can be hastened by oven drying, for example, at about 40.degree. C. for some 90 minutes. The thickness of the coating, regardless of coating method used, is preferably about 0.1 mil.

Alternatively, the rate of release of metal ions can be adjusted by using multiple coating layers characterized by differing carboxylic-acid components. A first layer, applied as described above, can thus incorporate a water-insoluble carboxylic acid and a second, overlying layer a water-soluble carboxylic acid. In such an arrangement, there is an initial high rate of release of metal ions from the latter layer, as the water-soluble carboxylic acid does not affect the antimicrobial activity of the metal-containing sulfonylurea. The release from the underlying layer, on the other hand, is slower, due to the presence of the water-insoluble carboxylic acid, which effects long-term release.

The user of an antimicrobial pressurized gas container according to a preferred embodiment of the present invention is human. However, any other mammals may be users of such inventive gas containers. Exemplary mammals include, but are not limited to, dogs, cats, cows, horses, rats, mice, monkeys, and rabbits.

Antimicrobial treatment of pressurized gas containers may also involved the induction of mutation to block colonization by microbes. Mutations can arise spontaneously as a result of events such as errors in the fidelity of DNA replication or the movement of transposable genetic elements (transposons) within the genome. They also are induced following exposure to chemical or physical mutagens. Such mutation-inducing agents include ionizing radiations, ultraviolet light and a diverse array of chemical such as alkylating agents and polycyclic aromatic hydrocarbons all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation. Mutation also can be site-directed through the use of particular targeting methods.

In alternative embodiments according to the present invention, chemical mutagenesis offers certain advantages, such as the ability to find a full range of mutant alleles with degrees of phenotypic severity, and it is facile and inexpensive to perform. The majority of chemical carcinogens produce mutations in DNA. Benzo[a]pyrene, N-acetoxy-2-acetyl aminofluorene and aflotoxin B1 cause GC to TA transversions in bacteria and mammalian cells. Benzo[a]pyrene also can produce base substitutions such as AT to TA. N-nitroso compounds produce GC to AT transitions. Alkylation of the 04 position of thymine induced by exposure to n-nitrosoureas results in TA to CG transitions.

In other alternative embodiments according to the present invention, the integrity of biological molecules may be degraded by the ionizing radiation. Adsorption of the incident energy may lead to the formation of ions and free radicals, and breakage of some covalent bonds. Susceptibility to radiation damage appears quite variable between molecules, and between different crystalline forms of the same molecule. It depends on the total accumulated dose, and also on the dose rate (as once free radicals are present, the molecular damage they cause depends on their natural diffusion rate and thus upon real time). Damage is reduced and controlled by making the sample as cold as possible.

In addition to providing an antimicrobial surface for a gas container as shown in FIG. 1, and as discussed above, other embodiments according to the present invention may also incorporate similar or other antimicrobial coatings or agents in the valves, connectors, regulators, and other flow-through components which attach to such gas containers in their various applications. FIG. 2 shows additional details for an exemplary gas flow regulator which may be provided with antimicrobial linings, coatings, or inherent properties in any or all of its components. The exemplary gas regulator of FIG. 2 shows a valve 145 attached to a gas tank 110 at tank junction 135. The exemplary gas regulator of FIG. 2 is further provided with a pressure gauge 140 and a gas outlet 150. In various embodiments according to the present invention, any or all of the components shown in FIG. 2 may be provided with antimicrobial coatings, linings, or fabricated of inherently antimicrobial materials, using the coating or fabrication materials and methods previously described for the provision of antimicrobial properties with the interior of a gas container according to the present invention.

Finally, while there have been shown and described and pointed out fundamental novel features of the present invention as applied to preferred embodiments thereof, it will be understood that various omissions and substitutions and changes in the materials, form, and details of the devices and processes illustrated, and in their operation, and in the method illustrated and described, may be made by those skilled in the art without departing from the spirit of the invention as broadly disclosed herein. All of the above-discussed patents and publications are hereby expressly incorporated by reference as if they were written directly herein.

1-12. (canceled)

13. A method of retarding or preventing the colonization of microbes within interior wall surfaces of a tank or interior wall surfaces of a flow-through component for the containment and dispensing of pressurized gases by applying an antimicrobial surface therewithin.

14. The method of claim 13, wherein said antimicrobial surface is a coating.

15. The method of claim 13, wherein said antimicrobial surface comprises an antiseptic.

16. The tank of claim 13, wherein said antimicrobial surface comprises an antibiotic.

17. The tank of claim 13, wherein said antimicrobial surface comprises one or more antimicrobial agents in effective concentrations to decrease, prevent, or inhibit growth of bacterial and/or fungal organisms within said tank.

18. The tank of claim 13, wherein said interior wall surfaces comprise a coating with inherent antimicrobial properties.
